(19) United States
(12) Patent Application Publication
(10) Pub. No.: US 2022/0143401 A1
(43) Pub. Date: May 12, 2022

(54) Earthing Acupressure Device

(71) Applicant: Jeremy Madvin, Westlake Village, CA (US)

(72) Inventor: Jeremy Madvin, Westlake Village, CA (US)

(21) Appl. No.: 17/095,772

(22) Filed: Nov. 12, 2020

(51) Int. Cl. A61N 1/14; A61H 39/04

(52) U.S. Cl. CPC A61N 1/14; A61H 2201/0153; A61H 2201/10; A61H 39/04

(57) ABSTRACT

The present invention discloses an enhanced design of acupressure device which can use both techniques simultaneously posing a tremendous opportunity to increase the results of a typical acupressure session through dramatically increased, targeted and controlled ion passage through and around the meridian point, enhancing the effects of grounding through allowing for a pinpointed ion gateway and also effective in potentiating their biological and clinical benefits, while reducing costs at the same time.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates to an acupressure pen and methods of acupressure treatment, wherein said pen is capable of combining the principles of earthing with the principles of targeted acupressure.

### Description of the Related Art

Acupressure is one of the popular CAM (complementary and alternative medicine) techniques around the world. It is based on the fundamental concept of the activation of acupoints around the meridians. The activation of acupressure points is enabled by the use of fingers or the various hand-held acupressure devices that are easily accessible. Acupressure is practiced by many individuals around the world due to the simple and reliable manual (hands-on) therapy.

Incorporating grounding with acupressure using a specialized probe makes it possible to precisely target the area of a specific meridian point. The user can choose the path and exit point for the body's excess voltage and direct it though specific meridians to increase stimulation and assist in the unblocking process.

Acupressure is a superior therapeutic technique for various disease disorders. Acupressure is an acupuncture type. Both acupressure and acupuncture are based on the same basic concept of meridian-acupoint stimulation. Muscular stress is released in acupressure by applying hand pressure at specific acupoints or thumb pressure at specific points or by applying pressure to acupoints to control the physiological energy flow, Acupressure demands the application of physical pressure at trigger points/acupoints/specific pressure points located along the meridians.

Meridians are the human body channels that help to sustain Qi and therefore maintain health conditions. Each meridian is linked to multiple human body organs and tissues. Pressure activation of a particular point on a meridian promotes pain relief at the local site and also decreases pain from other areas of the body. It is a pressure point, a hand-mediated energy healing technique that, along with beneficial physical effects, satisfaction, and economy, is recognized as a useful tool for treating different symptoms. To facilitate the well-being of patients, acupressure is a manually controlled, needle-free, non-invasive, cost-effective, and non-pharmacological healing intervention.

The human body is a cluster of pressure points. An acupoint is the point nearest to the surface of the skin and the first step of acupressure is the activation of the acupoint.6 Stimulation of identified acupoints is known to induce functional responses that can be used to treat diseases. Acupoints are any particular points of acupressure for needle insertion; There are often areas of high electrical conductance on the surface of the body. Every acupoint has a different sensation depending on the part of the body that suffers from pain or a particular problem. Simply stated, various effects are caused by the application of pressure at various points.

Acupressure transfers pleasurable impulses to the brain four times faster than unpleasant stimulation at a specific stage. Continuous impulses close down the neural ‘GATES’ and slower pain signals are blocked from accessing the brain and help to boost or increase the body's pain tolerance threshold. Pressure generation stimulates the small myelin nerves in the muscles depending on the stimulus position and transmits stimuli to the higher nerve centers, including the spinal cord, midbrain, hypothalamus, and pituitary. Magnetoencephalography (MEG) studies demonstrated the conditioning during acupressure of the primary somatosensory response. In addition to the sub-cortical impact on endogenous pain management, acupressure often can influence primary somatosensory perception and likely change maladaptive neuroplasticity. 10 Auricular acupressure (AA) is a non-invasive technique similar to reflexology in which endocrine functions are effectively enhanced. It is believed to exert its effects on the auricle of the ear by activating the central nervous system via the cranial/spinal nerves.

There are multiple inventions that can be seen in prior art in similar regards. For instance, a Smart acupressure pen and methods of administering acupressure bearing U.S. Pat. No. 2,009,0076422A1 is issued to Hong Kong Polytechnic University HKPU. The present invention relates to an acupressure pen and methods of acupressure treatment, wherein said pen is capable of determining the optimal range for treatment on a particular acupoint and recalling such optimal range during subsequent treatments. Through the use of a grid system, infrared communicator, and microcontroller, the pen is capable of being set and storing results in a matrix.

Another patent on Needling device for acupuncture bearing Chinese patent 2,073,748U is issued to Chinese inventor. The utility model relates to a needling device for acupuncture, belonging to the field of the Chinese medicine clinic acupuncture application. The utility model is characterized in that the needling device for acupuncture comprises a sleeve tribe which is composed of a pen-shaped needling end 1, a round pipe 2 and a firing end 7, and a firing system which is composed of a spring 6, a thin copper needing holding reed 3, a needling pipe 4, a firing bolt 8 and a firing switch 5. The firing end is provided with an F-shaped port, and the firing switch is arranged in the F-shaped port. The utility model changes the traditional acupuncture needling method; the needle puncturing is quick, accurate and axenic; the patient has no pain feeling. The utility model has the advantages of simple production process, low cost and convenient operation, and can be widely used for the Chinese medicine clinic acupuncture.

Another patent on Acupuncturing needles bearing Chinese patent 2,688,286Y is issued to Chinese inventor. The utility model relates to an acupuncturing needle which can be inserted through a cannula, comprising a cannula and an acupuncturing needle arranged in the inner cavity of the cannula. One end of the cannula is provided with a screw propulsive device, one end of the acupuncturing needle is arranged in the screw propulsive device, and the screw propulsive device is arranged in the cannula. One end of the acupuncturing needle is arranged in the screw propulsive device, the top part of the screw propulsive device is provided with a handle used for rotation, the other end of the cannula is provided with a pore for threading the acupuncturing needle, the end part of the cannula is provided with a shim, and the screw propulsive device is provided with a ruler. When the screw propulsive device is propelled along the cannula, the acupuncturing needle can be inserted in an acupoint accurately.

Another patent on Opto-mechatronic acupressure pen bearing U.S. Pat. No. 2,006,0270956A1 is issued to Hong Kong Polytechnic University HKPU. The patent is on an acupuncture pen comprises: a casing (11); a blunt tip (13), provided at the front end of the casing movably; a sensing unit (12), provided in the casing (11) for detecting the movement of the tip (13); An A/D converter (15), provided in the casing (11) for converting the analog signal from the sensing unit (12) into digital signal; a microcontroller (16), provided in the casing (11) for receiving the digital signal from the A/D converter (15), and calculating the force blunt tip (13) applied on the skin on the basis of the received digital signal; and a digital display, provided in the wall of the casing, and electrically connected with the microcontroller (16) for indicating the calculated force. With such structure, the acupuncture pen of this invention can achieve compact configuration and indicate the force applied by the pen visually.

Another patent on Device for manual application of acupressure bearing U.S. Pat. No. 5,560,746A is issued to Sky F. Willow. The patent is on a manual device for self-application of acupressure by a user, comprises a substantially “J”-shaped rigid frame having a first tip at one end for application of pressure to specific points of the user's body and a handle at the other end. The first tip is adapted to be placed on the back portions of the user while the frame is substantially transverse to the user's body. The handle is adapted to be grabbed by the user such that a pushing force imparted to the handle away from the user's body transmits pressure to the first tip.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease of use ability of these assemblies is also questionable.

It has to be noted that the current invention proposes an assembly with multiple advancements including a structure with symmetrical measurements in height and side width that make it more stable and easier to use and carry. It promotes a technology where the device could be used by both techniques simultaneously and poses a tremendous opportunity to increase the results of a typical acupressure session through dramatically increased, targeted and controlled ion passage through and around the meridian point, and enhances the effects of grounding through allowing for a pinpointed ion gateway and might be effective in potentiating their biological and clinical benefits, while reducing costs at the same time. The assembly is made of high quality metal, alumina or some other electrically conductive material with a narrow tip not intended to pierce the skin.

None of the previous inventions and patents, taken either singly or in combination, are seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of an acupressure and grounding simple, non-invasive and accessible techniques in single unit.

It is another objective of the invention to provide an acupuncture device which being used is increasingly agreeable for the patient.

It is further the objective of the invention to acupressure and grounding are simple, non-invasive, and accessible techniques that when allows the regulation or treatment of inflammation, pain, circulation, and autonomic dysfunctions.

It is also the objective of the invention to provide a device of the type described which provides variety of relaxation techniques, exercises and stretches that assist to restore and recover problematic soft tissues in the body.

It is also the objective of the invention to provide a device which increases the results of a typical acupressure session through dramatically increased, precisely targeted and controlled ion passage through and around the meridian point.

It is further the objective of the invention to restore issues associated with pain enhancing the effects of grounding through allowing for a pinpointed ion gateway.

It is also the objective of invention which is also effective in potentiating its biological and clinical benefits, while reducing costs at the same time.

It is also the objective of invention to provide a device which is comprised of a handheld probe, and a wire or some other physical conduit to an electrical outlets grounding port.

It is another objective of the invention to provide a device that is crafted from very firm metal, alumina or some other electrically conductive material with a narrow tip not intended to pierce the skin.

It is moreover the objective of the invention to provide assembly which is a simple but efficient manual device for self-stimulation by acupressure on points on the skin that are difficult to access, like the back, the back part of the muscles, bottom of feet, etc., without the subject having to be in inappropriate body positions. The invention permits completion of an acupressure technique of catalytic medicine, in the same way.

A further object is to provide an assembly that is simple, and easy to use.

A still further object is to provide an assembly that is economical in cost to manufacture.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a full view of the invention.

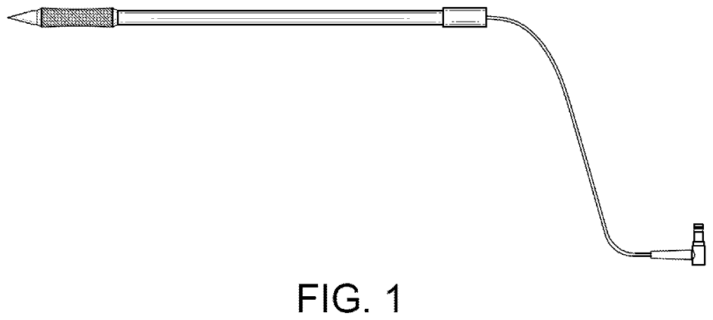

FIG. 2 shows another view of the invention.

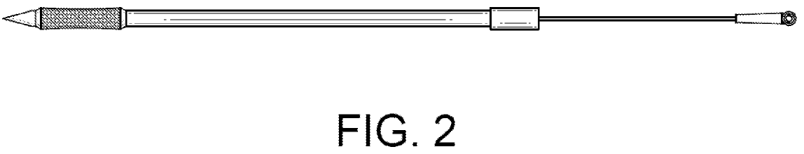

FIG. 3 shows wire at the end.

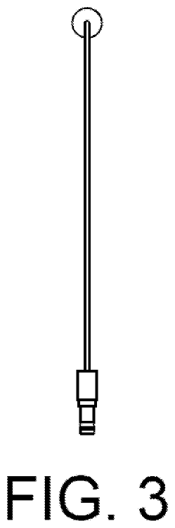

FIG. 4 shows environmental view.

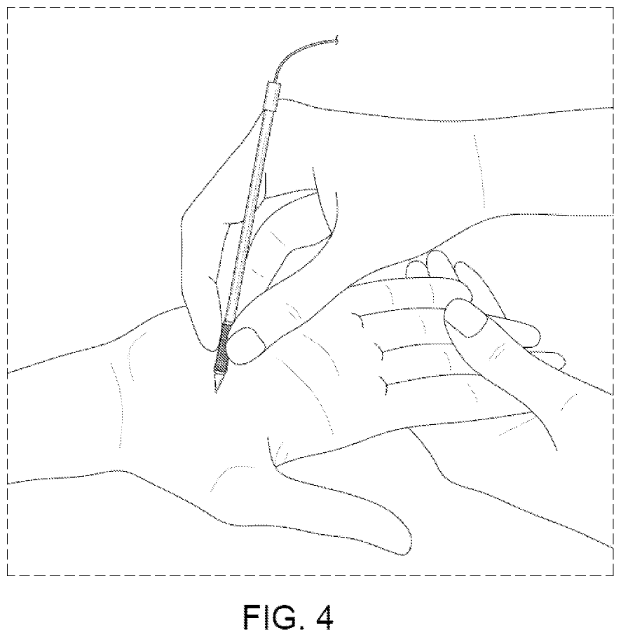

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to an Earthing Acupressure Device which presents principles of earthing with the principles of targeted acupressure.

This device is designed to apply mechanical pressure while simultaneously accepting a large volume of electrons from a specific location on the body. It is designed to then transfer these electrons to an indoor grounding outlet receptacle where they can then be deposited into the earth.

Acupressure transfers pleasurable impulses to the brain four times faster than unpleasant stimulation at a specific stage. Continuous impulses close down the neural ‘GATES’ and slower pain signals are blocked from accessing the brain and help to boost or increase the body's pain tolerance threshold. Pressure generation stimulates the small myelin nerves in the muscles depending on the stimulus position and transmits stimuli to the higher nerve centers, including the spinal cord, midbrain, hypothalamus, and pituitary. Magnetoencephalography (MEG) studies demonstrated the conditioning during acupressure of the primary somatosensory response. In addition to the sub-cortical impact on endogenous pain management, acupressure often can influence primary somatosensory perception and likely change maladaptive neuroplasticity.10 Auricular acupressure (AA) is a non-invasive technique similar to reflexology in which endocrine functions are effectively enhanced. It is believed to exert its effects on the auricle of the ear by activating the central nervous system via the cranial/spinal nerves.

The biochemical acupressure process as per its preferred embodiments includes the stimulation of acupoints leading to complex neurohormonal responses. This includes the counteraction between the hypothalamic-pituitary-adrenocortical axis leading to cortisol overproduction and triggering a reaction to relaxation. It also modulates the physiological response by increasing the delivery of endorphin and serotonin through nerves and meridians to the brain and specific organs. Restorative meridian acupressure helps to adjust the concentration of stress hormones and lactic acid after exercise. Specifically in the case of the respiratory function, acupressure plays a moderating role. Activation of acupoint activates myelinated neural fibers that stimulate the hypothalamus and pituitary gland, leading to the release of β-endorphins from the hypothalamus into the spinal fluid and pituitary into the bloodstream, respectively. Thus, the analgesic and sedative effect of β-endorphins facilitates the normal respiratory function of the patient.

The traditional Earthing means reconnecting the conductive human body to the normal and subtle electrical surface charge of the Earth, an effortless lifestyle practice that systemically affects the body's fundamental bioelectrical function. Doing so stabilizes the physiology remarkably, decreases inflammation, pain, and stress, increases sleep, blood flow, and lymphatic/venous return to the heart, and creates greater well-being. There are structural consequences that are profound. People also say that they feel and look safer and younger after they begin Earthing. Many with pain experience less pain. Even morale improves. Earthing is simple to do and, especially for people with chronic health problems, often produces fast results.

As per its further embodiments there are two fundamental forms of Earthing: 1) indoors, involving inexpensive Earthing systems while sifting or sleeping (conductive mats, sheets, body bands, or patches), and 2) in its most natural form, and free, by spending time outdoors barefoot in contact with natural conductive surfaces, such as grass, dirt, gravel, stone, and sand.

Earthing, due to the human lifestyle, restores a primordial electrical link to the planet lost over time. Earthing seems to correct what we call an “electron deficiency syndrome”, an overlooked and possibly main cause of different health circumstances.

Earthing is a powerful method for preventing and rehabilitating health and healing, a technique that can be used on its own or in combination with clinical therapies to alleviate not only symptoms but root causes of symptoms and dysfunction that begin at the physiology's deep bioelectric levels.

It is now time for the medical community to begin to understand that a body attached to the ground is equally more secure and healthier than a ungrounded body. It works more naturally, as humans have become increasingly detached from the Planet, a condition lost over time. Obviously, we no longer sleep on the grass, rarely walk outside barefoot, and almost exclusively wear insulating plastic sole shoes for more than half a century instead of conventional and conductive leather footwear. We live and work, spending most or some of our time disconnected, often in high rises well above ground.

Thus the lack of contact with the Earth can lead to electrical imbalances, the accumulation of destructive static electricity (positive charges), and an unrecognized deficit of electrons in the body, as well as susceptibility to malfunction, disease, and disorder. As is well known, Planet Earth's surface is electrically influenced by countless lightning bolts, solar radiation, and other dynamics of the atmosphere. However, it is not so well understood that these phenomena give our land masses and water bodies a continually refreshed supply of subatomic particles called free electrons that give the Earth a normal negative electrical charge. Biological Earthing research indicates that for both the animal and plant kingdoms, this very same electrical charge, and its infinite supply of electrons and their diurnal frequencies, plays a governing and nurturing function, a form of “electrical nutrition,” so to speak. The research results have endorsed the hypothesis that Earthing enables a substantial transfer of free electrons into the body, resulting in rapid physiological changes, often instantaneous. Earthing preserves and retains an internal electrical condition that is normal. A few years ago, Sokal and Sokal, two Polish doctors and scholars, conducted a series of experiments and concluded that the human body is a “universal controlling force in Nature” Bio electric, bioenergetic, and biochemical processes are highly regulated and tend to have a significant modulating effect on chronic diseases and dysfunction.

As per its further embodiments the external biological stimulation triggers electrical and chemical reactions that influence important biological activities in human life. In Oriental medicine, acupressure is also considered to induce such reactions, thereby allowing unbalanced physiological situations to return to normal balanced physiological conditions. In Western medicine, the physiological theory of acupressure has been explained as a mechanism for cytology-based nerve and endocrine cell control. The sensory system is activated by the sensory or nerve receptor neural complex in the process of acupressure. However, while acupressure stimulation results in brain responses, it may not be accurate for all acupressure responses to occur only through the nervous system, because the biological response to external stimulation through the nervous system is much faster than is possible. This indicates that biological systems other than the nervous system could be involved in acupressure reactions. As a result, acupressure may be called the movement of electrical energy into nervous or connective tissue.

It has been stated that acupressure stimulation appears to produce ion-electric-charge, which is transmitted via various biological pathways. It is also well known that the effect of acupressure therapy depends on the electrical charge volume or the duration of acupressure stimulation. Regarding electricity, the acupressure stimulation of the Meridian circuit amplifies the small currents and voltages that this circuit produces. This electricity can result from the electrical potential due to the current produced by the stimulation of acupressure points by the acupressure needle or the chemical reaction between the acupuncture needle and the hypodermis. As it affects the distribution of ions and bioelectrical charges, the chemical reaction can be presumed to have the same effect as the electrical reaction.

Meridians may be known to be ion-electric-charge pathways. Blocking the ion-electric-charge results in an unbalanced negative and positive charge in a meridian, and hence pain and the generation of various diseases. Acupressure moves the excessive or insufficient state of the ion-electric-charge to allow good health to be restored. This suggests that acupuncture is closely linked to the transfer of bioelectrical charges. However, acupuncture based on meridian theory does not necessarily lead to consistent effective therapeutic outcomes. However, results in multiple therapeutic outcomes, namely acupressure and grounding provide an increased likelihood of effect with no potential consequences.

Acupressure has been widely used as a primary therapeutic technique due to its minimal side-effects and therapeutic efficacy. However, under conditions of acupressure that may affect the efficacy of acupressure therapy, the grounded response to acupressure stimulation has not been explicitly studied. To achieve the synergistic effect, both strategies are used together in new worlds. This technique is creating a revolution in acupressure research. You know that small e-stim tools can be used by your acupuncturist to inject electrons into your body. With excessive load, we can target very precise areas that are as small as scars that can cause problems or harm to a specific part of the body.

The assembly as per its preferred embodiments is a handheld acupressure probe that when applied to the skin, grounds the body upon connection through a single and precise point.

For achieving above object, this invention provides an acupuncture pen, which comprises: casing; a blunt tip, provided at the front end of the casing movably; a sensing unit, provided in the casing for detecting the movement of the tip; an A/D converter, provided in the casing for converting the analog signal from the sensing unit into digital signal; a microcontroller, provided in the casing for receiving the digital signal from the A/D converter, and calculating the force blunt tip applied on the skin on the basis of the received digital signal; and a digital display, provided in the wall of the casing, and electrically connected with the microcontroller for indicating the calculated force. Furthermore, the device is comprised of a handheld probe, and a wire or some other physical conduit to an electrical outlets grounding port.

The invention combines the ancient knowledge of traditional Chinese medicine with a sophisticated opto-mechatronic circuit, and makes use of a non-contact technique, i.e., infrared technique, to convert displacement data into a series of force signals. Therefore, it is convenient to use with the aid of the digital display of the applied force.

Incorporating grounding with acupressure using a specialized probe makes it possible to precisely target the area of a specific meridian point. The user can choose the path and exit point for the body's excess voltage and direct it though specific meridians to increase stimulation and assist in the unblocking process.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. An acupuncture pen comprising:
a casing;
a blunt tip, provided at he front end for movement;
a design further comprises of design to apply mechanical pressure while simultaneously accepting a large volume of electrons from a specific location on the body, It is designed to then transfer these electrons to an indoor grounding outlet receptacle where they can then be deposited into the earth.
